(19) United States
(12) Patent Application Publication
(10) Pub. No.: US 2022/0054300 A1
(43) Pub. Date: Feb. 24, 2022

(54) NON-INVASIVE DEVICE FOR CONTROLLING SLEEP APNEA AND SNORING

(71) Applicant: Hrushikesh VAIDYA, Thane (IN)

(72) Inventor: Hrushikesh VAIDYA, Thane (IN)

(21) Appl. No.: 17/417,058

(22) PCT Filed: Dec. 18, 2019
(86) PCT No.: PCT/IN2019/050931

§ 371 Date: Jun. 21, 2021

(30) Foreign Application Priority Data
Dec. 19, 2018 (IN) 201821048156

(51) Int. Cl. A61F 5/56

(52) U.S. Cl. CPC A61F 5/56

(57) ABSTRACT

The present disclosure describes a non-invasive device (100) for controlling sleep apnea and snoring. The non-invasive device (100) comprises a sub-mandibular support member (102) adapted to fit a mandible of a user; a chest support member (110) adapted to rest on a chest of the user; a pair of spring elements (116-1, 116-2) coupled to the sub-mandibular support member (102) and the chest support member (110) and in communication with the sub-mandibular support member (102), the pair of spring elements (116-1, 116-2) adapted to be in an expanded position when the mandible is in a forward position and in a compressed position when the mandible is in a normal position; and a strap member (124) adapted to support the sub-mandibular support member (102) and the chest support member (110) on the user such that the strap member (124), the chest support member (110), and the sub-mandibular support member (102) are adapted to restrict a movement of the pair of spring elements (116-1, 116-2) in an upward direction when the pair of spring elements (116-1, 116-2) is in the compressed position; and wherein the sub-mandibular support member (102) is adapted to move the mandible in a forward direction from the normal position to the forward position such that an airway opening is created and maintained when the pair of spring elements (116-1, 116-2) is in the compressed position.

### TECHNICAL FIELD

The present disclosure generally relates to a medical device for treating sleep disorders, and in particular, to a non-invasive device for controlling sleep apnea and snoring.

### BACKGROUND

Obstructive sleep apnea hypopnea syndrome (OSAHS or simply, obstructive sleep apnea, hereinafter referred to as “sleep apnea”) has become a common and serious medical disorder in today's fast paced and hectic lifestyles. The disorder is characterized by recurrent sleep induced collapse of the pharyngeal airway, which leads to hypoxaemia and hypercapnia with arousal from sleep being required to re-establish airway patency. Sleep apnea decreases oxygen levels in the body resulting in daytime fatigue, cardiovascular problems, and, in some cases, may even result in death. It has been suggested that patients with obstructive sleep apnea have anatomical predisposition to airway collapse. As such, controlling sleep apnea and snoring have gained importance and relevance.

Various mechanisms or therapies are available to treat obstructive sleep apnea. Such mechanisms include straps, dental devices, continuous positive airway pressure, and surgical procedures. However, these mechanisms are invasive in nature, i.e., require introduction of devices or objects into the body or body cavities. This causes a lot of inconvenience or uncomfortable to the patient, thereby leading to ineffective patient compliance. To overcome such problems, non-invasive devices are available in the market for controlling sleep apnea and snoring. However, such non-invasive devices are costly and complex in nature. In addition, the patient has to manually perform various steps to use such non-invasive devices for getting effective results. Thus, there is a need for a non-invasive device that is simple to use and controls sleep apnea and snoring.

### SUMMARY

This summary is provided to introduce a selection of concepts in a simplified format that are further described in the detailed description of the present disclosure. This summary is not intended to identify key or essential inventive concepts of the claimed subject matter, nor is it intended for determining the scope of the claimed subject matter. In accordance with the purposes of the disclosure, the present disclosure as embodied and broadly described herein describes a non-invasive device for controlling sleep apnea and snoring.

In accordance with embodiments of the present disclosure, a non-invasive device for controlling sleep apnea and snoring is disclosed. The non-invasive device comprises a sub-mandibular support member, a chest support member, a pair of spring elements, and a strap member. The sub-mandibular support member is adapted to fit a mandible of a user. The chest support member is adapted to rest on a chest of the user. The pair of spring elements is coupled to the sub-mandibular support member and the chest support member and is in communication with the sub-mandibular support member. The pair of spring elements is adapted to be in an expanded position when the mandible is in a forward position and in a compressed position when the mandible is in a normal position. The strap member is adapted to support the sub-mandibular support member and the chest support member on the user. The strap member, the chest support member, and the sub-mandibular support are adapted to restrict a movement of the pair of spring elements in an upward direction when the pair of spring elements is in the compressed position. When the pair of spring elements is in the compressed position, the sub-mandibular support member is adapted to move the mandible in a forward direction from the normal position to the forward position such that an airway opening is created and maintained.

The advantages of the present invention include, but not limited to, providing a simple and non-expensive non-invasive device for non-invasively opening up user's airway thereby controlling sleep apnea and snoring by way of minimizing the occurrence of sleep apnea and snoring. The device may prevent the sleep apnea and snoring due to continuous use over a period of time.

These and other features, aspects and advantages of the present disclosure will become better understood with reference to the following drawings, description and claims.

### BRIEF DESCRIPTION OF FIGURES

These and other features, aspects, and advantages of the present invention will become better understood when the following detailed description is read with reference to the accompanying drawings in which like characters represent like parts throughout the drawings, wherein:

FIG. 1 illustrates a perspective view of a non-invasive device for controlling sleep apnea and snoring, in accordance with an embodiment of the present disclosure; and

FIG. 2A and FIG. 2B illustrate side views of the non-invasive device attached to a user, in accordance with the embodiments of the present disclosure.

Further, skilled artisans will appreciate that elements in the drawings are illustrated for simplicity and may not have been necessarily been drawn to scale. For example, the flow charts illustrate the method in terms of some operations involved to help to improve understanding of aspects of the present disclosure. Furthermore, in terms of the construction of the device, one or more components of the device may have been represented in the drawings by conventional symbols, and the drawings may show some specific details that are pertinent to understanding some example embodiments of the inventive concepts so as not to obscure the drawings with details that will be readily apparent to those of ordinary skill in the art having benefit of the description herein.

### DETAILED DESCRIPTION

For the purpose of promoting an understanding of the principles of the disclosure, reference will now be made to some example embodiments illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates. Unless otherwise defined, all technical and scientific terms used herein have the same meaning as commonly understood by one of ordinary skilled in the art to which this disclosure belongs. The system, methods, and examples provided herein are illustrative only and not intended to be limiting. Embodiments of the present disclosure will be described below in detail with reference to the accompanying drawings.

Embodiments of the present disclosure will be described below in detail with reference to the accompanying drawings.

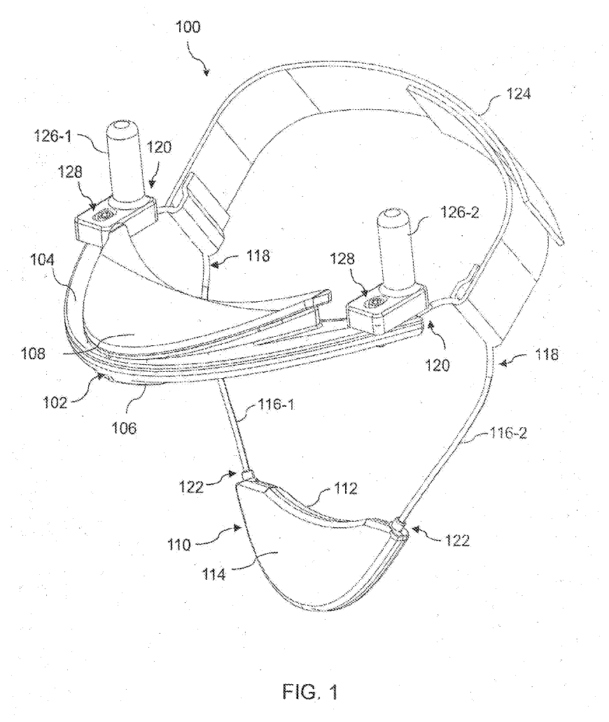

FIG. 1 illustrates a perspective view of a non-invasive device 100 for controlling sleep apnea and snoring, in accordance with an embodiment of the present disclosure. The non-invasive device 100 (hereinafter referred to as “the device 100”) is adapted to non-invasively open up an airway of a user thereby controlling sleep apnea and snoring. In an implementation, the sleep apnea and snoring are controlled by way of minimizing the occurrence of sleep apnea and snoring.

In accordance with the embodiment, the device 100 comprises sub-mandibular support member 102 adapted to fit a mandible of a user. The sub-mandibular support member 102 has a large surface area to adequately and appropriately support under-chin of the user. The sub-mandibular support member 102 may be a single elongated curved structure that is shaped to fit with the mandible and the under-chin of the user. The sub-mandibular support member 102 includes an upward surface or user-facing surface 104 and a downward surface or outward facing surface 106. The user-facing surface 104 is adapted to receive the mandible and the under-chin of the user patient, and the outward facing surface 106 faces away from the user. In some example implementation, the sub-mandibular support member 102 may be sized and shaped to fit the mandible and/or the under-chin of the user. The sub-mandibular support member 102 can be made in a single layer construction from materials including but not limited to polyurethane (PU) foam, Ethylene-vinyl acetate (EVA) foam, or similar soft materials adaptable to provide both rigid support and comfort while wearing.

The sub-mandibular support member 102 comprises a padding element 108 to cushion the mandible and the under-chin of the user while wearing. The padding element 108 is lined or affixed to the user-facing surface 104 and therefore is sized and shaped in conformance with the user-facing surface 104. The padding element 108 can be made of any soft padding material that is well tolerated by skin of the user and provide comfort while wearing. Examples of such padding material include, but not limited to, silicone gel, soft polyurethane, EVA foam, PU foam, and other suitable material. In an implementation, the padding element 108 may have an optional adhesive coating or lining that can adhere to the skin of the user in sub-mandibular region of the user without causing irritation to the skin when used for long duration. The padding element 108 can be lined or affixed to the user-facing surface 104 using techniques or mechanisms as known in the art.

Further, the device 100 comprises a chest support member 110 adapted to rest on a chest of the user. The chest support member 110 has a large surface area such that the chest support member 110 can adequately and appropriately rest on chest of the user. The chest support member 110 may be a single elongated curved or curvilinear structure. The chest support member 110 includes an inward surface or user-facing surface 112 and an outward facing surface 114. The user-facing surface 112 is shaped to correspond to a shape of outer surface of the user in an upper chest area. For example, in some implementations, the user-facing surface 112 is adapted to comfortably rest on the body of the user in proximal to collar bones without the interfering with protrusions of the collar bones. The outward facing surface 114 faces away from the user. In some example implementation, the chest support member 110 may be sized and shaped to fit the upper chest of the user. The chest support member 110 can be made in a single layer construction from materials including but not limited to polyurethane (PU) foam, Ethylene-vinyl acetate (EVA) foam, or similar soft materials adaptable to provide both rigid support and comfort while wearing.

The chest support member 110 comprises a padding element (not shown in the figure) to cushion the chest while wearing. The padding element is lined or affixed to the user-facing surface 112 and therefore is sized and shaped in conformance with the user-facing surface 112. The padding element can be made of any soft padding material that is well tolerated by skin of the user and provide comfort while wearing. Examples of such padding material include, but not limited to, silicone gel, soft polyurethane, EVA foam, PU foam, and other suitable material. In an implementation, the padding element may have an optional adhesive coating or lining that can adhere to the skin of the user or clothes worn by the user without causing irritation to the skin or damage to the clothes when used for long duration. The padding element can be lined or affixed to the user-facing surface 112 using techniques or mechanisms as known in the art.

Further, the device 100 comprises a pair of spring elements 116-1, 116-2 coupled to the sub-mandibular support member 102 and the chest support member 110 to connect the sub-mandibular support member 102 with the chest support member 110. Each of the pair of spring elements 116-1, 116-2 is an elongated structure with a bend 118 defining a specific angle (hereinafter referred as “the spring angle”). The pair of spring elements 116-1, 116-2 is adapted to maintain between the sub-mandibular support member 102 and the chest support member 110 and a specific stiffness. The pair of spring elements 116-1, 116-2 can be made from a metallic material such as stainless steel or spring steel.

Further, each of the pair of spring elements 116-1, 116-2 is coupled to the sub-mandibular support member 102 at one end via a connecting member 120 and is coupled to the chest support member 110 at other end via a connecting member 122. The pair of spring elements 116-1, 116-2 is releasably coupled to the sub-mandibular support member 102 and the chest support member 110. Such releasably coupling of the pair of spring elements 116-1, 116-2 allows for customization of one or more parameters of the pair of spring elements 116-1, 116-2 to accommodate variations in the anatomical dimensions of different users. The parameter of the pair of spring elements 116-1, 116-2 includes the spring angle, the stiffness, bend radius, bend arc, length, breadth/width, and thickness. In an example, the pair of spring elements 116-1, 116-2 is in the form of a wire. In such example, in one implementation, thickness of the wire can be smaller such that the pair of spring elements 116-1, 116-2 can have a low stiffness. In such example, in another implementation, thickness of the wire can be larger such that the pair of spring elements 116-1, 116-2 can have a high stiffness. In another example, the pair of spring elements 116-1, 116-2 has a long length to accommodate users having longer necks. In another example, the pair of spring elements 116-1, 116-2 has a short length to accommodate users having shorter necks.

Now, the pair of spring elements 116-1, 116-2 is adapted to be in an expanded position when the mandible is in a forward position in relation to a movement of a neck of the user in an upward direction. The forward position can be a position in which the neck is moved upwards such as in a chin-up pose. The pair of spring elements 116-1, 116-2 is adapted to be in a compressed position when the mandible is in a normal position in relation to a movement of the neck of the user in a downward direction. The normal position can be a relaxed sleeping position such as side sleeping position or back sleeping position in which the neck is moved downwards towards the chest.

Further, the device 100 comprises a strap member 124 adapted to support the sub-mandibular support member 102 and the chest support member 110 on the user. The strap member 124 is adapted to adjustably support the neck of the user such that a movement of the sub-mandibular support member 102 and the chest support member 110 is restricted in relation to movement of the user. This results in securely and firmly holding the device 100 in its position while wearing. As such, the strap member 124 can include, but not limited to, Velcro mechanism, hook & loop mechanism, tape mechanism. In an implementation, ends of the strap member 124 are releasably coupled at to the pair of spring elements 116-1, 116-2 at the bend 118, as shown in the FIG. 1. In an implementation, ends of the strap member 124 are releasably coupled to the chest support member 110. In such implementation, the chest support member 110 includes a pair of holders for coupling the ends of the strap member 124 with the holders. In both implementations, the strap member 124, the chest support member 110, and the sub-mandibular support member 102 are adapted to restrict a movement of the pair of spring elements 116-1, 116-2 in an upward direction when the pair of spring elements 116-1, 116-2 is in the compressed position.

Further, the device 100 comprises a pair of mandible supports 126-1, 126-2 coupled to the sub-mandibular support member 102. Each of the pair of mandible supports 126-1, 126-2 is adapted to adjustably fit a posterior angle of the mandible at each side of the mandible. Each of the pair of mandible supports 126-1, 126-2 is coupled to the sub-mandibular support member 102 using a locking member 128. Examples of the locking member 128 include but not limited to nut and bolt, screws and caps, etc. The pair of mandible supports 126-1, 126-2 is releasably coupled to the sub-mandibular support member 102. Such releasably coupling of the pair of mandible supports 126-1, 126-2 allows for customization of pair of mandible supports 126-1, 126-2 to accommodate variations in the mandibular dimensions of different users.

Further, each of the pair of mandible supports 126-1, 126-2 has lining of a padding element (not shown in the figure) to cushion the mandible of the user while wearing. The padding element can be made of any soft padding material that is well tolerated by skin of the user and provide comfort while wearing. Examples of such padding material include, but not limited to, silicone gel, soft polyurethane, EVA foam, PU foam, and other suitable material.

Thus, the device 100 is a modular structure such that the aforementioned different parts of the device 100 can be easily removed and replaced as necessary. The device 100 is able to properly fit the user and effectively control sleep disorder by a combination of various aspects provided in the device 100, i.e., (a) secure resting of the sub-mandibular support member 102 and the chest support member 110; (b) provision for adjusting the strap member 124; (c) provision for adjusting the pair of mandible supports 126-1, 126-2; and (d) provision for adjusting the pair of spring elements 116-1, 116-2 to adjust parameters such as the spring angle and the spring stiffness.

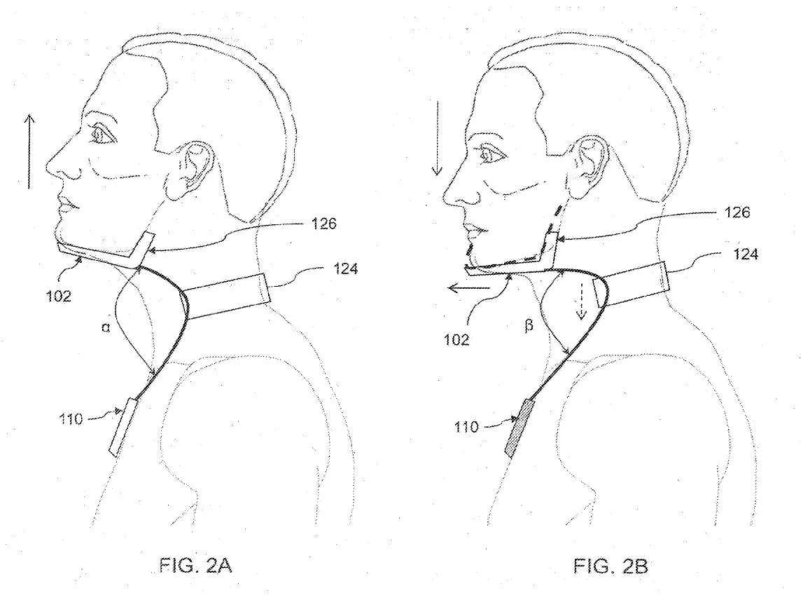

FIG. 2A and FIG. 2B illustrate side views of the non-invasive device attached to the user, in accordance with the embodiment of the present disclosure. Referring to FIG. 2A, the mandible of the user is initially in the normal position. A movement of the neck in slight extension position or in upward direction (represented by up arrow) results in the movement of the mandible to a forward position or protruded with varied degrees. The device 100 is then worn by the user such that the sub-mandibular support member 102 and the pair of mandible supports 126-1, 126-2 are fit to the mandible, and the chest support member 110 is rested on the chest. The strap member 124 is sufficiently tightened such that the chest support member 110 is firmly rested on the chest; the sub-mandibular support member 102 has firm contact with the sub-mandibular skin and the pair of mandible supports 126-1, 126-2 just touch the posterior of the mandible. At this position, i.e., while the user is wearing the device 100, the device 100 is in initial position or natural free position and the pair of spring elements 116-1, 116-2 is in the expanded position or natural free position. The pair of spring elements 116-1, 116-2 is adapted to form a first spring angle (α) in the expanded position in accordance with the bend 118.

Referring to FIG. 2B, upon wearing the device 100, the neck is moved in downward direction (represented by down arrow) in relaxed position. This movement results in the movement of the mandible to the normal position. This leads to a load or force being exerted on the pair of spring elements 116-1, 116-2, thereby causing a movement of the pair of spring elements 116-1, 116-2 from the expanded position to the compressed position at the bend 118 in downward direction (represented by dashed down arrow). The pair of spring elements 116-1, 116-2 is adapted to form a second spring angle (α′ or β) in the compressed position.

At the compressed position, the pair of spring elements 116-1, 116-2 is adapted to move to the expanded position in upward direction due to the spring action caused by the bend 118. However, the movement of the pair of spring elements 116-1, 116-2 in the upward direction is restrained by the strap member 124, the chest support member 110, the sub-mandibular support member 102, and the pair of mandible supports 126-1, 126-2. This leads to a movement of the pair of spring elements 116-1, 116-2 in forward direction thereby causing the sub-mandibular support member 102 to move the mandible in a forward direction (represented by dashed line) from the normal position to the forward position. The movement of the mandible exerts a force in forward direction on tissues in submandibular region of the user (represented by horizontal directional arrow) to create the airway opening. This results in tensioning of the genioglossus and geniohyoid muscles which maintains the airway opening and aids in preventing sleep apnea. Further, the force is transferred to tongue and hyoid bone of the user such that the hyoid bone is pushed in the forward direction to increase the opening of the airway. This may result in preventing severe sleep apnea.

Thus, the airway opening of the user is created and retained non-invasively. This results in minimizing the occurrence of sleep apnea and snoring, thereby controlling the sleep apnea and snoring. The device 100 may prevent the sleep apnea and snoring due to continuous use over a period of time.

While specific language has been used to describe the present disclosure, any limitations arising on account thereto, are not intended. As would be apparent to a person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. Clearly, the present disclosure may be otherwise variously embodied, and practiced within the scope of the claims that follow.

1. A non-invasive device (100) for controlling sleep apnea and snoring, the non-invasive device (100) comprising:
a sub-mandibular support member (102) adapted to fit a mandible of a user;
a chest support member (110) adapted to rest on a chest of the user;
a pair of spring elements (116-1, 116-2) coupled to the sub-mandibular support member (102) and the chest support member (110) and in communication with the sub-mandibular support member (102), the pair of spring elements (116-1, 116-2) adapted to be in an expanded position when the mandible is in a forward position and in a compressed position when the mandible is in a normal position; and
a strap member (124) adapted to support the sub-mandibular support member (102) and the chest support member (110) on the user such that the strap member (124), the chest support member (110), and the sub-mandibular support member (102) are adapted to restrict a movement of the pair of spring elements (116-1, 116-2) in an upward direction when the pair of spring elements (116-1, 116-2) is in the compressed position; and
wherein the sub-mandibular support member (102) is adapted to move the mandible in a forward direction from the normal position to the forward position such that an airway opening is created and maintained when the pair of spring elements (116-1, 116-2) is in the compressed position.

2. The non-invasive device (100) as claimed in claim 1, wherein the movement of the mandible exerts a force in forward direction on tissues in submandibular region of the user to create and maintain the airway opening.

3. The non-invasive device (100) as claimed in claim 1, wherein:
the pair of spring elements (116-1, 116-2) is in the expanded position when the mandible is in the forward position in relation to a movement of a neck of the user in an upward direction; and
the pair of spring elements (116-1, 116-2) is in the compressed position when the mandible is in the normal position in relation to a movement of the neck of the user in a downward direction.

4. The non-invasive device (100) as claimed in claim 1, wherein the pair of spring elements (116-1, 116-2) is adapted to form a first spring angle (α) in the expanded position and a second spring angle (α′) in the compressed position, wherein the first spring angle is greater than the second spring angle.

5. The non-invasive device (100) as claimed in claim 1, wherein:
the sub-mandibular support member (102) comprises a padding element (108) to cushion the mandible; and
the chest support member (110) comprises a padding element to cushion the chest.

6. The non-invasive device (100) as claimed in claim 1, wherein:
the sub-mandibular support member (102) is to have a shape and a size adapted to fit the mandible and an under-chin region of the user; and
the chest support member (110) is to have a shape and a size adapted to securely rest on the chest.

7. The non-invasive device (100) as claimed in claim 1, wherein the strap member is adapted to adjustably support a neck of the user such that a movement of the sub-mandibular support member (102) and the chest support member (110) is restricted in relation to movement of the user.

8. The non-invasive device (100) as claimed in claim 1, wherein the strap member is coupled to one of the pair of spring elements (116-1, 116-2) and the chest support member (110).

9. The non-invasive device (100) as claimed in claim 1 comprises a pair of mandible supports (126-1, 126-2) coupled to the sub-mandibular support member (102) and is adapted to adjustably fit a posterior angle of the mandible at each side of the mandible.

10. The non-invasive device (100) as claimed in claim 8, wherein the pair of mandible supports (126-1, 126-2) is adapted to restrict the movement of the pair of spring elements (116-1, 116-2) when the pair of spring elements (116-1, 116-2) is in the compressed position and to move the mandible in the forward direction from the normal position to the forward position such that the airway opening is created and maintained when the pair of spring elements (116-1, 116-2) is in the compressed position.
